(19) United States
(12) Patent Application Publication
(10) Pub. No.: US 2005/0129652 A1
(43) Pub. Date: Jun. 16, 2005

(54) Use of native zein for improving the condition of hair, and corresponding agent

(76) Inventors: Walter Keller, Ober Ramstadt (DE); Karl-Heinz Kischka, Darmstadt (DE)

Correspondence Address: Striker Striker & Stenby, 103 East Neck Road, Huntington, NY, 11743, US

(21) Appl. No.: 10/501,163

(22) PCT Filed: Sep. 26, 2003
(86) PCT No.: PCT/EP03/10747

(51) Int. Cl. A61K 7/06; A61K 7/11

(52) U.S. Cl. 424/70.14

(57) ABSTRACT

The object of the present invention is the use of natural zein for improving the condition of hair, preferably for hardening, strengthening, restructuring or increasing the luster, volume or combability of human hair.

The object of the present invention is the use of natural zein for improving the condition of hair, preferably for hardening, luster improvement, strengthening and structure improvement (restructuring) or increasing the volume of hair, particularly damaged human hair.

It is known that keratin fibers are damaged by environmental influences (for example high-energy radiation), physiological status (for example age or health of the individual involved) or mechanical or chemical effects. This results in impairment of the mechanical properties of the affected materials. Such damage to the inner structure of keratin fibers is indicated, for example, by a loss of hardness, luster, breaking strength, tensile strength or bundle tensile strength.

On keratin fibers, particularly human hair, such effects are indicated especially by an absence of luster, reduced tensile strength and poor combability. Such effects are caused by aging processes induced primarily physiologically or as a result of physical (weathering), mechanical (combing, brushing) or chemical action. Long hair shows these effects particularly at the hair tips. Chemical effects include primarily bleaching, oxidative dyeing and permanent waving of hair, for which aggressive oxidants or reducing agents are used and even preferably in a strongly alkaline medium wherein they show their full effect. Keratin-containing material, however, can also be adversely affected by other chemical influences, for example by water enriched in chlorine or salts.

Commercial rinses and treatments contain as active substances mainly cationic surfactants or polymers, waxes and/or oils. The more damaged the hair, the more anionic groups are present on the hair surface. Cationic compounds are electrostatically attracted by this oppositely charged surface, whereas oils and waxes interact with the hydrophobic groups of keratin. An improvement in internal hair structure can therefore not be achieved with these hair-care agents.

The use of certain unsaturated compounds, particularly ascorbic acid, in hair-treatment agents for this purpose is known from our own WO 00/57839. Ascorbic acid, however, is not stable in aqueous solution for a long time, so that such agents cannot be stored, but must be prepared shortly before use.

U.S. Pat. No. 2,383,990 discloses the use of natural zein as film former or suspending agent and suspending coating material in a cosmetic preparation, for example for pigments that are insoluble in aqueous-alcoholic solution. In that patent, natural zein is also described as a film-forming polymer (hair coating) in agents for temporary shaping of hair, such as styling agents and hair setting, waving and/or curling agents. U.S. Pat. No. 2,383,990 thus discloses the use of natural zein in agents for transient shaping of hair, agents that contain no aggressive hair-damaging chemicals. What this patent does not disclose, however, is the use of natural zein for improving the condition of hair in terms of hardening, strengthening, restructuring, repairing or stabilizing hair or for increasing the luster, volume or combability of hair.

The purpose underlying the present invention was to provide a cosmetic hair-treatment agent for use in improving the condition of hair and which would eliminate the aforesaid drawbacks.

According to the invention, this objective is reached by use of natural zein for improving the condition of hair, preferably for hardening, strengthening, restructuring, repairing or stabilizing keratin fibers or for increasing the luster, volume or combability of keratin fibers.

For purposes of the invention, by natural zein is meant an unhydrolyzed protein obtained from corn (Zea mays), for example, by known extraction methods or commercially, for example by Sigma or Fluka.

The new teachings according to the invention were arrived at only by the inventors' discovery that natural zein brings about the hardening, strengthening, restructuring, repair or stabilization of hair and that it is suited for increasing the luster, volume or combability of hair—a fact established only after lengthy measurements of tensile strength and combing force.

Only the teachings of the present invention, however, would induce those skilled in the art to use natural zein for the applications of the invention, namely in hair-treatment agents that usually do not contain film-formers or in which film-formers are commonly not used or are even undesirable, namely in permanent wave agents or in agents for oxidative treatment of hair. Only after having learned about the new effects of natural zein would those skilled in the art use natural zein—completely in contrast to previous procedures—in agents the use of which has a particularly pronounced damaging effect on the hair so that the hair would require urgent repair. Such damaging agents are hair-treatment agents containing aggressive substances, such as permanent wave agents (which contain hair keratin-reducing mercaptans) or agents for oxidative treatment of hair, such as permanent wave neutralizing agents, blonding agents, hair bleaching agents and oxidative hair colorants (which contain persulfate or hydrogen peroxide). On the other hand, according to U.S. Pat. No. 2,383,990, hair is not at all damaged by styling agents or by styling agents that impart color by means of pigments.

Surprisingly, we have now found that by use of natural zein the structure of keratin fibers (hair) is modified so as to bring about hardening and strengthening as well as an increase in breaking strength, tensile strength or bundle tensile strength, particularly in case of weakened or damaged keratin fibers. Besides a hair-care effect resulting from action on the hair surface (cuticula), to be described in the following, we observed, in particular, a repair effect. This is attributable to changes inside the hair (cortex). We measured the tensile forces that cause previously oxidatively damaged hair (by bleaching) to break. Surprisingly, we found that hair which after the oxidative damage had been treated with an agent containing natural zein showed a significant increase in forces needed to cause breaking.

This is surprising, because on the basis of the structure of natural zein it was not possible to anticipate penetration into the hair nor any influence on the protein structure. Moreover, it is known that undamaged wet hair requires markedly lower breaking forces (600-900 mN) than does dry hair (1000-1500 mN). Hence, it was to be expected that a spheroprotein such as natural zein would reduce the breaking forces rather than increase them, as we have found.

In connection with this, not only is restructuring (repair) of damaged keratin fibers made possible, but a protective effect is exerted that counteracts damage caused to these materials before or during exposure to the said noxae and thus prevents or reduces such damage.

In addition to favorably affecting these deleterious changes caused by exogenous noxae, the use according to the invention can also bring about advantageous effects on conditions or changes in structure of keratin fibers brought about by physiological processes, for example, in the case of brittle hair caused by age or of congenital or age-related fine hair (baby hair, aged hair)

Furthermore, in this regard we found in keratin fibers, particularly hair, that by the use according to the invention it is possible to achieve an increase in volume which is advantageous in hair styling (increased hair fullness). It is suspected that the effect of the volume increase is in a causal relationship with the hair-hardening, hair-strengthening or hair-structuring effect of the agents used according to the invention.

Hence, the object of the present invention is the use according to claim 1.

Other embodiments of the present invention are covered by the other claims.

Natural zein is a constituent of corn protein to an extent of about 40% and is contained in corn gluten to an extent of about 60-70%.

The use consists of bringing the keratin fibers in contact with an agent containing natural zein, and after the application either leaving the agent on the fibers or after an appropriate exposure time rinsing it out or washing it out with an aqueous agent.

Preferably, the agent contains the natural zein in an amount from 0.001 to 20.0 weight percent, preferably from 0.01 to 10.0 weight percent and particularly from 0.05 to 3.0 weight percent, based on the total amount of the agent.

The agent described for the use according to the invention can be present in all suitable formulations known in the cosmetic or pharmaceutical industry. In particular, the agent can be in the form of an aqueous or aqueous-alcoholic solution, or of a gel, cream, emulsion or foam and it can be packaged in the form of a one-component preparation or in the form of a multicomponent preparation. In the case of a one-component preparation, the agent contains the natural zein together with appropriate auxiliary agents and carriers (for example thickeners, acids, fragrances, solvents, salts, wetting agents and/or UV absorbers).

If the agent is in the form of a multicomponent preparation, it can consist of at least two different components that are kept spatially separated until the time of use. The first component can either contain the natural zein underlying the present invention (active substance) alone, or the active substance can be present in this first component mixed together with an auxiliary agent (for example a thickener), preferably in dry, solid form (for example as a powder in compressed or uncompressed form, as a granulate or as a tablet). A second component or the additional components contain only auxiliary agents or carriers.

It is also possible, however, that in a multicomponent preparation different components contain different active substances according to the present invention alone or as a mixture, either alone or together with different auxiliary agents, and that the other components contain only auxiliary agents and carriers.

The invention comprises the use of a composition characterized in that said composition is a one-component preparation or a multicomponent preparation. If the agent used is a multicomponent preparation, then it comprises a first component containing the natural zein with or without auxiliary agents and additives and a second component containing the other ingredients. The agent used can also be a multicomponent preparation with at least three different components, in which case at least one of the components contains the natural zein and the other components contain the other ingredients.

Naturally, to prepare a ready-for-use agent, the individual spatially separated components of a multicomponent preparation must be mixed shortly before they are used according to the invention.

The agent used according to the invention can additionally contain carriers and auxiliary agents, for example a solvent such as water, a lower aliphatic alcohol, for example ethanol, n-propanol or isopropanol, a glycol ether or a glycol such as glycerol and particularly 1,2-propanediol; moreover dissolution promoters, wetting agents or emulsifiers from the classes of anionic, cationic, amphoteric or nonionic surface-active substances such as the fatty alcohol sulfates, ethoxylated fatty alcohol sulfates, alkylsulfonates, alkylbenzenesulfonates, alkyltrimethylammonium salts, alkylbetaines, ethoxylated fatty alcohols, ethoxylated nonylphenols and fatty alkanolamides, furthermore thickeners such as the higher fatty alcohols, starch or cellulose derivatives; salts such as, for example, NaCl; buffering substances such as ammonium hydrogen carbonate; thiols, ketocarboxylic acids (oxocarboxylic acids), particularly c-ketocarboxylic acids or their physiologically tolerated salts, UV absorbers, perfumes, dyes, conditioners, hair swelling agents, preservatives, vaselines, paraffin oil and fatty acids as well as hair-care agents such as cationic resins, lanolin derivatives, cholesterol, pantothenic acid and betaine, and propellants such as, for example, propane, butane, dimethyl ether, N₂O and carbon dioxide. The aforesaid constituents are used in amounts commonly employed for such purposes, for example water in an amount from 0.1 to 95 wt. %, the wetting agents and emulsifiers at a total concentrations from 0.2 to 30 weight percent, the alcohols in a total amount from 0.1 to 90 weight percent, the opacifying agents, perfume oils, preservatives and dyes in an amount from 0.01 to 5 wt. % each, the buffering substances in a total amount from 0.1 to 10 weight percent and the dissolution promoters, stabilizers, hair-conditioning and hair-care constituents in an amount from 0.1 to 5 weight percent each, whereas the thickeners and dissolution promoters can be contained in said agent in a total amount from 0.5 to 20 weight percent.

The pH of the agent is preferably 2.0 to 10.0 and particularly 3.0 to 9.0. If necessary, the pH can be adjusted to the desired value by addition of an acid, for example an α-hydroxycarboxylic acid such as lactic acid, tartaric acid, citric acid or malic acid, phosphoric acid, acetic acid, glycolic acid, salicylic acid, glutathione or gluconolactone, or with an alkalinizing agent such as ammonia, an alkanolamine, alkylamine, alkali metal hydroxide, ammonium hydroxide, alkali metal carbonate, ammonium carbonate or alkali metal phosphate.

In the treatment of keratin fibers, the agent can be left on the fibers (for example on the hair) or it can be rinsed out after use. In the latter case, the time of exposure to the agent is from 1 to 60 minutes and particularly from 5 to 20 minutes, depending on the temperature (about 20 to 60° C. and preferably from 30 to 50° C.), it being possible to accelerate the repairing action (hardening, restructuring and optionally the volume increase associated therewith) by supplying heat; hence, the use of heat is preferred. At the end of the exposure time, the hair can be rinsed with water and optionally washed with a shampoo.

The formulations suitable for the use of the agent according to the invention are preferably oxidative hair dyes, hair bleaching agents, oxidative hair-tinting lotions, hair-deforming agents such as permanent wave agents or hair defrizzing agents or hair-stretching agents and hair-smoothing agents or fixing agents. Preferred is the use in hair treatment agents that have a weaking effect on the hair caused by the action of their chemical ingredients, particularly reducing agents, oxidants or strong bases.

For this reason, in an advantageous embodiment of the invention, natural zein is used in an oxidative hair colorant, preferably in an amount from 0.01 to 10 wt. % and particularly in an amount from 0.1 to 5 wt. %.

In this case, the hair colorant contains (A) natural zein and (B) one or more oxidation dye precursors and/or one or more direct natural or synthetic dyes. Suitable oxidation dye precursors that produce the coloration by action of an oxidant, for example hydrogen peroxide or in the presence of atmospheric oxygen, are, for example, the following developers and couplers and self-coupling compounds.

(i) Developers

1,4-Diaminobenzene (p-phenylenediamine), 1,4-diamino-2-methylbenzene (p-toluylenediarnine), 1,4-diamino-2,6-dimethylbenzene, 1,4-diamino-3,5-diethylbenzene, 1,4-diamino-2,5-dimethylbenzene, 1,4-diamino-2,3-dimethylbenzene, 2-chloro- 1,4-diaminobenzene, 1,4-diamino-2-(thiophen-2-yl)benzene, 1,4-diamino-2-(thiophen-3-yl)benzene, 4-(2,5-diaminophenyl)-2-[(diethylamino)methyl]thiophene, 2-chloro -3-(2,5-diaminophenyl)thiophene, 1,4-diamino-2-(pyridin-3-yl)benzene, 2,5-diaminobiphenyl, 2,5-diamino4′-(1-methylethyl)-1,1′-biphenyl, 2,3′,5-triamino-1,1′-biphenyl, 1,4-diamino-2-methoxymethylbenzene, 1,4-diamino-2-aminomethylbenzene, 1,4-diamino-2-[(phenylamino)methyl]benzene, 1,4-diamnino-2-{[ethyl-(2-hydroxyethyl)amino]methyl}benzene, 1,4-diamino-2-hydroxymethylbenzene, 1,4-diamino-2-(2-hydroxyethoxy)benzene, 2-[2-(acetylamino)ethoxy]-1,4-diaminobenzene, 4-phenylaminoaniline, 4-dimethylaminoaniline, 4-diethylaminoaniline, 4-dipropylaminoaniline, 4-[ethyl-(2-hydroxethyl)amino]aniline, 4-[di(2-hydroxyethyl)amino]aniline, 4-[di(2-hydroxyethyl)amino]-2-methyl-aniline, 4-[(2-methoxyethyl)amino]aniline, 4-[(3-hydroxypropyl)amino]aniline, 4-[(2,3-dihydroxy-propyl)amino]aniline, 4-{[(4-aminophenyl)methyl]amnino}aniline, 4-[(4-aminophenylamino)methyl]-phenol, 1,4-diamino-N-(4-pyrrolidin-1-ylbenzyl)benzene, 1,4-diamino-N-furan-3-ylmethylbenzene, 1,4-diamino -N-thiophen-2-ylmethylbenzene, 1,4-diamino-N-furan-2-ylmethylbenzene, 1,4-diamino-N-thiophen-3-ylmethylbenzene, 1,4-diamino-N-benzylbenzene, 1,4-diamino-2-(1-hydroxyethyl)benzene, 1,4-diamino -2-(2-hydroxyethyl)benzene, 1,4-diamino-2-(1-methylethyl)benzene, 1,3-bis[(4-aminophenyl)-(2-hydroxyethyl)amino]-2-propanol, 1,4-bis[(4-aminophenyl)amino]butane, 1,8-bis(2,5-diaminophenoxy)-3,6-dioxaoctane, 2,5-diamino4′-hydroxy-1,1′-biphenyl, 2,5-diamino-2′-trifluoromethyl-1,1′-biphenyl, 2,4′,5-triamino-1,1′-biphenyl, 4-amninophenol, 4-amino-3-methylphenol, 4-amino-3-(hydroxymethyl)phenol, 4-amino-3-fluorophenol, 4-methylaminophenol, 4-amino-2-(aminomethyl)phenol, 4-amino-2-(hydroxymethyl)phenol, 4-amino-2-fluorophenol, 4-amino-2-[(2-hydroxyethyl)amino]-methylphenol, 4-amino-2-methylphenol, 4-amino-2-(methoxymethyl)phenol, 4-amino-2-(2-hydroxy-ethyl)phenol, 5-amninosalicylic acid, 2,5-diaminopyridine, 2,4,5,6-tetraaminopyrimidine, 2,5,6-tri-amino-4-(1H)-pyrimidone, 4,5-diamnino-1-(2-hydroxyethyl)-1H-pyrazole, 4,5-diamino-1-(1-methyl-ethyl)-1H-pyrazole, 4,5-diamino-1-[(4-methylphenyl)methyl]-1H-pyrazole, 1-[(4-chlorophenyl)methyl]-4,5-diamino-1H-pyrazole, 4,5-diamino-1-methyl-1H-pyrazole, 4,5-diamino-1-pentyl-1H-pyrazole, 4,5-diamino-1-(phenylmethyl)-1H-pyrazole, 4,5-diamino-1-[(4-methoxyphenyl)methyl-1H-pyrazole, 2-aminophenol, 2-amino-6-methylphenol, 2-amino-5-methylphenol, 1,2,4-trihydroxybenzene, 2,4-di-aminophenol, 1,4-dihydroxybenzene and 2-{([(4-aminophenyl)amino]methyl}-1,4-diaminobenzene, alone or in admixture with one another.

(ii) Couplers

N-(3-Dimethylaminophenyl)urea, 2,6-diaminopyridine, 2-amino4-[(2-hydroxyethyl)amino]anisole, 2,4-diamino-1-fluoro-5-methylbenzene, 2,4-diamino-1-methoxy-5-methylbenzene, 2,4-diamino-1-ethoxy-5-methylbenzene, 2,4-diamino-1-(2-hydroxyethoxy)-5-methylbenzene, 2,4-di[(2-hydroxyethyl)-amino]-1,5-dimethoxybenzene, 2,3-diamino-6-methoxypyridine, 3-amino-6-methoxy-2-(methylamino)-pyridine, 2,6-diamino-3,5-dimethoxypyridine, 3,5-diamino-2,6-dimethoxypyridine, 1,3-diaminobenzene, 2,4-diamino-1-(2-hydroxyethoxy)benzene, 1,3-diamino-4-(2,3-dihydroxypropoxy)benzene, 1,3-diamino-4-(3-hydroxypropoxy)benzene, 1,3-diamino4-(2-methoxyethoxy)benzene, 2,4-diamino-1,5-di(2-hydroxyethoxy)benzene, 1-(2-aminoethoxy)-2,4-diaminobenzene, 2-amino-1-(2-hydroxyethoxy)-4-methylaminobenzene, 2,4-diaminophenoxyacetic acid, 3-[di(2-hydroxyethyl)amino]aniline, 4-amino-2-di[(2-hydroxyethyl)amino]-1-ethoxybenzene, 5-methyl-2-(1-methylethyl)phenol, 3-[(2-hydroxyethyl)amino]anilione, 3-[(2-aminoethyl)amino]aniline, 1,3-di(2,4-diaminophenoxy)propane, di(2,4-diaminophenoxy)methane, 1,3-diamino-2,4-dimethoxybenzene, 2,6-bis-(2-hydroxyethyl)aminotoluene, 4-hydroxyindole, 3-dimethylaminophenol, 3-diethylaminophenol, 5-amino-2-methylphenol, 5-amino-4-fluoro-2-methylphenol, 5-amino4-methoxy-2-methylphenol, 5-amino4-ethoxy-2-methylphenol, 3-amino-2,4-dichlorophenol, 5-amnino-2,4-dichlorophenol, 3-amino-2-methylphenol, 3-amino-2-chloro-6-methylphenol, 3-aminophenol, 2-[(3-hydroxyphenyl)amino]acetamide, 5-[(2-hydroxyethyl)amino]-4-methoxy-2-methylphenol, 5-[(2-hydroxyethyl)amino]-2-methylphenol, 3-[(2-hydroxyethyl)amino]-phenol, 3-[(2-methoxyethyl)amino]phenol, 5-amino-2-ethylphenol, 5-amino-2-methoxyphenol, 2-(4-amino-2-hydroxyphenoxy)ethanol, 5-[(3-hydroxypropyl)amino]-2-methylphenol, 3-[(2,3-dihydroxypropyl)amino]-2-methylphenol, 3-[(2-hydroxyethyl)amino]-2-methylphenol, 2-amino-3-hydroxypyridine, 2,6-dihydroxy-3,4-dimethylpyridine, 5-amino-4-chloro-2-methylphenol, 1-naphthol, 2-methyl-1-naphthol, 1,5-dihydroxynaphthalene, 1,7-dihydroxynaphthalene, 2,3-dihydroxynaphthalene, 2,7-dihyrdoxynaphthalene, 2-methyl-1-naphthol acetate, 1,3-dihydroxybenzene, 1-chloro-2,4-dihydroxybenzene, 2-chloro-1,3-dihydroxybenzene, 1,2-dichloro-3,5-dihydroxy4-methylbenzene, 1,5-dichloro-2,4-dihydroxybenzene, 1,3-dihydroxy-2-methylbenzene, 3,4-methylenedioxyphenol, 3,4-methylenedioxyaniline, 5-[(2-hydroxyethyl)amino]-1,3-benzodioxol, 6-bromo-1-hydroxy-3,4-methylenedioxybenzene, 3,4-diaminobenzoic acid, 3,4-dihydro-6-hydroxy-1,4(2H)-benzoxazine, 6-amino-3,4-dihydro-1,4(2H)benzoxazine, 3-methyl-1-phenyl-5-pyrazolone, 5,6-dihydroxyindole, 5,6-dihydroxyindoline, 5-hydroxyindole, 6-hydroxyindole, 7-hydroxyindole and 2,3-indolinedione, alone or in admixture with one another.

(iii) Self-coupling Compounds

2-Amino-5-methylphenol, 2-amino-6-methylphenol, 2-amino-5-ethoxyphenol or 2-propylamino-5-aminopyridine.

The oxidation dye precursors are present in the hair colorant in a total amount from about 0.01 to 12 weight percent and particularly from about 0.2 to 6 weight percent.

To achieve certain color shades, the hair colorant can also contain common natural or synthetic (nonoxidative) direct dyes, for example triphenylmethane dyes, aromatic nitro dyes, azo dyes, quinone dyes, cationic or anionic dyes, henna or indigo.

Moreover, the hair colorant can contain antioxidants, for example ascorbic acid, thioglycolic acid or sodium sulfite, as well as complexing agents for heavy metals, for example an ethylenediaminetetraacetate or nitriloacetic acid, in an amount of up to about 0.5 weight percent. Perfume oils can be present in the dye carrier composition in an amount of up to about I weight percent.

Naturally, the hair colorant can optionally contain other additives commonly used in hair colorants, for example preservatives and perfume oils; antioxidants, for example sodium sulfite, thioglycolic acid or ascorbic acid; complexing agents; solvents such as water, the lower aliphatic alcohols, for example ethanol, propanol and isopropanol, or glycols such as glycerol and 1,2-propylene glycol; wetting agents or emulsifiers from the classes of anionic, cationic, amphoteric or nonionic surface-active substances; thickeners such as the higher fatty alcohols, starch or cellulose derivatives; furthermore softeners, vaselines, silicone oils, paraffin oil and fatty acids, as well as cationic resins, for example combability improvers and anti-hair frizzing substances, such as cationic polymers for example CTTA: POLYQUATERNIUM-1, CTFA: POLYQUATERNIUM4, CTFA: POLYQUATERNIUM-5, CTFA: POLYQUATERNIUM-6, CTFA: POLYQUATERNIUM-7 CTFA: POLYQUATERNIUM-10, CTFA: POLYQUATERNIUM-11, CTFA: POLYQUATERNIUM-16, CTFA: POLYQUATERNIUM-22, CTFA: POLYQUATERNIUM-32, CTFA: POLYQUATERNIUM-35, CTFA: POLYQUATERNIUM-36, CTFA: POLYQUATERNIUM-37, CTFA: POLYQUATERNIUM-39, CTFA: POLYQUATERNIUM-44, CTFA: POLYQUATERNIUM45, CTFA: POLYQUATERNIUM-46, CTFA: POLYQUATERNIUM47, CTFA: POLYSILICONE-3, CTFA: POLYSILICONE-4, CTFA: POLYSILICONE-5, CTFA: POLYSILICONE-6, CTFA: POLYSILICONE-7, CTFA: POLYSILICONE-8 and CTFA: POLYSILICONE-1; lanolin derivatives, cholesterol, vitamins, pantothenic acid and betaine. The abbreviation “CTFA” stands for International Cosmetic Ingredient Dictionary and Handbook, eighth edition 2000 (ISBN 1-882621-22-0).

The said constituents are used in amounts commonly employed for such purposes, for example the wetting agents and emulsifiers at a concentration from 0.1 to 30 weight percent, the thickeners in an amount from 0.1 to 25 weight percent and the hair-care agents at a concentration from 0.1 to 5. 0 weight percent.

Particularly advantageous is the addition of nonionic and/or anionic surfactants or emulsifiers, for example fatty alcohol sulfates, particularly lauryl sulfate and sodium cocoylsulfate, ethoxylated fatty alcohol sulfates, particularly sodium lauryl ether sulfates with 2 to 4 ethylene oxide units in the molecule, ethoxylated fatty esters,, ethoxylated nonylphenols, ethoxylated fatty alcohols, alkylbenzenesulfonates or fatty alkanolamides, in a total amount from about 0.1 to 30 weight percent and preferably from 0.2 to 15 weight percent.

The pH of the hair colorant based on oxidation dye precursors is in the range from about 6 to 12 and preferably from 9 to 11, whereas the pH of the ready-for-use oxidation hair colorant (namely the mixture of the hair colorant of the invention and the oxidant) is from about 5.5 to 10 and preferably from 6 to 9. Depending on the composition and the desired pH of the hair colorant, the pH is adjusted preferably with ammonia or an organic amine, for example a glucamine, aminnomethylpropanol, monoethanolamine or triethanolamine or with an inorganic base, for example sodium hydroxide, potassium hydroxide, sodium carbonate or calcium hydroxide, or with an organic or inorganic acid, for example lactic acid, citric acid, acetic acid or phosphoric acid.

The oxidative hair colorant or oxidative hair tinting agent is preferably formulated as an aqueous or aqueous-alcoholic preparation, for example as a thickened solution or as an emulsion, cream or gel. Suitable alcohols are, in particular, the lower alcohols with 1 to 4 carbon atoms commonly used for cosmetic purposes, for example ethanol and isopropanol.

For use in oxidative dyeing, the afore-described hair colorant or hair tinting agent is mixed with an oxidant just before use, and the mixture is applied to the hair in an amount sufficient for the dyeing, as a rule about 60 to 200 grams of the ready-for-use preparation.

Suitable oxidants for developing the coloration are mainly hydrogen peroxide or the compounds of addition thereof to urea, melamine or sodium borate in the form of a 1 to 12% and preferably 1.5 to 6% aqueous solution. The mixing ratio of hair colorant to oxidant depends on the concentration of the oxidant and as a rule is from about 5:1 to 1:2 and preferably 1:1, the amount of oxidant in the ready-for-use preparation preferably being from about 0.5 to 8 weight percent and particularly from 1 to 4 weight percent.

The ready-for-use hair colorant is allowed to act on the hair at 15 to 50° C. for about 10 to 45 minutes and preferably for about 15 to 30 minutes, after which the hair is rinsed with water and dried. Optionally, following this rinsing the hair is washed with a shampoo and possibly post-rinsed with a weak organic acid, for example tartaric acid. The hair is then dried.

The natural zein can also be used in a pretreatment agent employed before a chemical and/or physical treatment of keratin fibers, particularly oxidative hair dyeing, oxidative hair tinting, hair bleaching or permanent hair deformation, so as to avoid hair damage by these oxidative treatments.

Hence, an advantageous embodiment of the invention concerns the use of natural zein, preferably in an amount from 0.01 to 10 wt. % and particularly in an amount from 0.1 to 5 wt. %, in a pretreatment agent, before the application of a cosmetic agent containing aggressive chemicals such as reducing agents or oxidants, and preferably in a pretreatment agent for permanent waving. The permanent wave pretreatment agent is brought in contact with the hair, particularly the hair tips, before the application of the permanent wave agent (reducing agent or oxidative fixative). The natural zein preferably seals the particularly damaged hair sites—especially the hair tips—thus protecting them from the action of the subsequently used reducing permanent wave agent.

In another embodiment, the natural zein is used in a permanent hair deforming agent (permanent wave agent or hair defrizzing agent) or in an agent for oxidative treatment of hair (for example, for bleaching, brightening or fixing the hair).

The permanent hair deforming agent then contains (A) natural zein, preferably in an amount from 0.01 to 10 wt. % and particularly in an amount from 0.1 to 5 wt. %, and (B) at least one hair keratin-reducing substance.

The active deforming substances that can be used in the hair-deforming agent are those based on common hair keratin-reducing substances, for example the salts of sulfurous acid or certain mercapto compounds, particularly the salts or esters of mercaptocarboxylic acids. The permanent hair-deforming agent contains the keratin-reducing compounds in amounts commonly employed for hair deformation, for example the ammonium salts of thioglycolic acid or thiolactic acid or cysteine, at a concentration from 6 to 12 weight percent. The pH of the alkaline deforming agent generally ranges from 7 to 10, the pH adjustment preferably being performed with ammonia, monoethanolamine, ammonium carbonate or ammonium hydrogen carbonate. If the deforming component is adjusted to an acidic pH (for example pH=6.5 to 6.9), the esters of mercaptocarboxylic acids, for example the glycol esters of monothioglycolic acid or the glyceryl esters of monothioglycolic acid, but preferably mercaptoacetamide or 2-mercaptopropionaride are used at a concentration from 2 to 14 weight percent, or the salts of sulfurous acid, for example sodium, ammonium or monoethanolammmonium sulfite, at a concentration from 3 to 8 weight percent (calculated as SO₂). The preferably used hair keratin-reducing compound is a salt or a derivative of a mercaptocarboxylic acid. The keratin-reducing compound is most preferably selected from among thioglycolic acid, cysteine and thiolactic acid or the salts thereof.

To enhance the effect of the permanent hair-deforming agent, it is possible to add to said agent a swelling agent or penetrant such as urea, a polyhydric alcohol, ether, melamine, alkali metal thiocyanate or ammonium thiocyanate, isopropanol, imidazolidin-2-one, 2-pyrrolidone and 1-methyl-2-pyrrolidone, at a concentration from about 0.5 to 50 weight percent and preferably from 2 to 30 weight percent.

After a length of exposure sufficient for permanent deformation of the hair and which amounts to 3 to 30 minutes and preferably 2 to 20 minutes depending on the hair condition, pH and deformation efficacy of the deforming agent as well as on the treatment temperature, the hair is oxidatively fixed, preferably without first rinsing the deforming agent out of the hair.

The agent for oxidative treatment of hair is characterized in that it contains (A) natural zein and (B) at least one oxidant.

Depending on the fullness of the hair, the said agent is used in an amount from about 50 to 200 g. The fixation can be carried out with any oxidant that has previously been used in fixatives. Examples of such oxidants are potassium bromate, sodium bromate, sodium perborate, dehydroascorbic acid, hydrogen peroxide and urea peroxide. The oxidant concentration varies depending on the duration of the application (as a rule from 1 to 40 minutes and preferably from 5 to 20 minutes) and on the application temperature (from 25 to 50° C.). In aqueous fixatives the oxidants are usually employed at a concentration from about 0.5 to 12.0 weight percent. The fixative can, of course, also contain other substances, for example weak acids or peroxide stabilizers.

Both the permanent hair-deforming agent and the agent for oxidative treatment of hair (for example the fixative) can be in the form of an aqueous solution or an emulsion or else in a water-based, thickened form, particularly a cream, gel or paste.

The fixative is most preferably in a viscous form with a consistency enabling the customer to have her hair fixed in the sitting position and not, as usual, by use of a wash basin. The fixative is preferably an oxidant-containing viscous preparation with a viscosity from 100 to 10,000 mPa.s at 25° C., a viscosity from 300 to 1000 mPa.s at 25° C. being particularly preferred. The viscosity data were obtained by measurements performed with a Haake rotational viscometer, model VT 501, at a shearing rate of 64.5 per second.

It is also possible to introduce these agents under pressure into aerosol tubes from which they can then be removed as aerosol foam.

Both the permanent hair-deforming agent and the agent for oxidative hair treatment can, of course, contain all additives usually employed and known for such agents, for example thickeners such as kaolin, bentonite, fatty acids, higher fatty alcohols, starch, polyacrylic acid, cellulose derivatives, alginates, vaseline or paraffin oil, wetting agents or emulsifiers from the classes of anionic, cationic, amphoteric or nonionic surface-active substances, for example fatty alcohol sulfates, fatty alcohol ether sulfates, alkylsulfonates, alkylbenzenesulfonates, quaternary ammonium salts, alkyl betaines, ethoxylated fatty alcohols, ethoxylated alkylphenols, fatty alkanolamides or ethoxylated fatty esters, furthermore opacifying agents, for example polyethylene glycol esters, or alcohols, for example ethanol, propanol, isopropanol or glycerol, dissolution promoters, stabilizers, buffering substances, perfume oils, hair conditioners, for example combability improvers, and anti-hair frizzing agents, such as cationic polymers, for example CTFA: POLYQUATERNIUM-1, CTFA: POLYQUATERNIUM-4, CTFA: POLYQUATERNIUM-5, CTFA: POLYQUATERNIUM-6, CTFA: POLYQUATERNIUM-7, CTFA; POLYQUATERNIUM-10, CTFA: POLYQUATERNIUM-11, CTFA: POLYQUATERNIUM-16, CTFA: POLYQUATERNIUM-22, CTFA: POLYQUATERNIUM-32, CTFA: POLYQUATERNIUM-35, CTFA: POLYQUATERNIUM-36, CTFA: POLYQUATERNIUM-37, CTFA: POLYQUATERNIUM-39, CTFA: POLYQUATERNIUM-44, CTFA: POLYQUATERNIUM-45, CTFA: POLYQUATERNIUM46, CTFA: POLYQUATERNIUM-47, CTFA: POLYSILICONE-3, CTFA: POLYSILICONE-4, CTFA: POLYSILICONE-5, CTFA: POLYSILICONE-6, CTFA: POLYSILICONE-7, CTFA: POLYSILICONE-8 and CTFA: POLYSILICONE-13; as well as hair-care constituents, for example lanolin derivatives, cholesterol, pantothenic acid, creatine or betaine. Moreover, optical brighteners in the form of derivatives of cumarin, stilbene, naphthalnimde, or benzoxazole or styryl derivatives can be added to these agents.

The said constituents can be used in amounts commonly employed for such purposes, for example the wetting agents and emulsifiers at a concentration from about 0.2 to 30 weight percent, and the thickeners in an amount from about 0.1 to 25 weight percent, in each case based on the ready-for-use agent.

The curlers are then removed, the fixative is rinsed out of the hair with water, and the hair is subjected to further treatment as usual. Advantageously, after the hair is subjected to permanent deformation, waves are created using water only.

We were able to establish that the use according to the invention of an agent containing natural zein in accordance with the present invention brings about a marked improvement in the structure of the previously damaged keratin fibers which can be confirmed by a statistically highly significant increase in tensile strength.

Measurement of the Repair Action

The determination of the tensile strength of hair, which is an indicator of the structural integrity of the hair cortex and thus a measure of the degree of damage, is carried out by tensile-elongation measurements commonly used for these purposes. From each hair strand, 20 individual hairs are selected, and the individual hair diameters are determined with a computer-controlled laser micrometer. By use of a tensile-elongation tester (MTT 160/600 Series Miniature Tensile Tester, Serial No. 600.95.05.001, supplied by DIA-STRON Ltd., England), a measurement is then made of the force required to cause the individual hairs to break. From these individual tensile stress values, which differ from one another because of the different hair diameters, the bundle tensile strength (BTS) is determined by calculating the tensile force and taking into account the hair diameters, for a hair diameter of 0.08 mm (average diameter). By taking into consideration the hair density, the conversion to units of bundle tensile strength (cN/tex) is finally performed. The higher the numerical value of the bundle tensile strength, the less pronounced is the hair damage.

Measurements on hair that had been treated with a natural zein-containing and a natural zein-free permanent wave pretreatment agent in the form of a shampoo gave the following results:

Damaged hair (blonded) was treated with the shampoo of Example 1 free of zein (the amount of natural zein was replaced with water): BTS: 10.51±0.4 cN/tex (determined on 18 hairs from a shampoo-treated strand);

Damaged hair (blonded) was treated with the shampoo of Example 1 containing 2 wt. % of natural zein: BTS=12.36±0.4 cN/tex (determined on 24 hairs from a shampoo-treated strand).

The difference between the above-indicated average values is statistically highly significant (significance level determined by the t-test: 99.9%). The 2 wt. % natural zein content increased the bundle tensile strength from 10.51 cN/tex to 12.36 cN/tex, corresponding to an increase of 17.6%. In other words, the addition of natural zein brought about a marked hair strengthening or repair.

Measurement of the Hair Combability Improvement

The combability of hair is also an important parameter for describing the hair quality. Various external factors, such as certain cosmetic treatments (bleaching, dyeing, permanent waving), weathering and frequent combing and brushing have a deleterious effect on the combability of hair that can be attributed to damage inflicted upon the cuticula.

The principle of most methods for measuring the combability is based on measuring the force (combing force) required to pass a comb through a hair strand under exactly defined boundary conditions.

Within the framework of our own studies, we used for this purpose an automated apparatus in which a mechanical gripper arm picked up the strands to be studied from a storage facility and hung them on the hook of a force-measuring unit. The strands were then combed several times automatically one after another at constant speed, and for each combing the combing force N (newton) was recorded as a function of the combing path (strand length). The indicated combing force values were finally obtained by averaging the combing force values over the combing path. The lower the combing force, the better is the combability of the hair.

Measurements on hair treated with and without natural zein-containing shampoo gave the following results.

Damaged hair (blonded) was treated with zein-free shampoo of Example 1 (the amount of natural zein was replaced with water). The combing force amounted to 1.43±0.05 N (measured average for three shampoo-treated hair strands).

Damaged hair (blonded) was treated with the shampoo of Example 1 containing 2 wt. % of natural zein: The combing force amounted to 1.21±0.08 N (measured average for three shampoo-treated hair strands).

The difference between the above-indicated average values is statistically significant (significance level determined by the t-test: 97.5%).

The 2 wt. % content of natural zein reduced the combing force from 1.43 N to 1.21 N, or by 15.3%. In other words, the addition of natural zein brought about a clearly detectable improvement in the wet combability of the hair.

Unless otherwise indicated, all percentages given in the present description are weight percentages, based on the total weight of a particular composition.

The subject matter of the invention will be explained in greater detail by way of the following examples.

### EXAMPLES

### Example 1

### Permanent Wave Pretreatment Agent (Shampoo)

| | | |
|---|---|---|
| Sodium lauryl ether sulfate (25% aqueous solution) | 40.0 | wt. % |
| Cocamidopropylbetaine | 5.0 | wt. % |
| NaCl | 2.0 | wt. % |
| Natural zein | 2.0 | wt. % |
| Water | to 100.0 | wt. % |

### Example 2

### Hair Shampoo for Permanent Wave Pretreatment

| | | |
|---|---|---|
| Sodium lauryl ether sulfate (25% aqueous solution) | 35.0 | wt. % |
| NaCl | 3.0 | wt. % |
| Triethanolamine | 4.0 | wt. % |
| 1,2-Dibromo-2,4-dicyanobutamine-2-phenoxyethanol | 0.1 | wt. % |
| Perfume oil | 0.1 | wt. % |
| Natural zein | 1.0 | wt. % |
| Water | to 100.0 | wt. % |

### Example 3

### Oxidation Hair Colorant in Cream Form

| | | |
|---|---|---|
| Stearyl alcohol | 8.000 | wt. % |
| Paraffin oil | 13.000 | wt. % |
| Wool grease | 6.000 | wt. % |
| Perfume | 0.300 | wt. % |
| 4-Aminophenol | 1.360 | wt. % |
| 1-Naphthol | 0.500 | wt. % |
| Resorcinol | 0.014 | wt. % |
| 2-Amino-6-chloro-4-nitrophenol | 0.003 | wt. % |
| Ammonia, 25% aqueous solution | 12.000 | wt. % |
| Disodium ethylenediaminetatraacetate | 1.000 | wt. % |
| Ascorbic acid | 1.000 | wt. % |
| Natural zein | 1.000 | wt. % |
| Water | to 100.000 | wt. % |

Just before use, 50 g of the foregoing hair colorant was mixed with 50 g of a 12% aqueous hydrogen peroxide solution. The mixture was then applied to blond natural hair and, after an exposure time of 30 min at 40° C., it was again rinsed out of the hair with water. After drying, the hair had a vivid copper-red color

### Example 4

### Permanent Wave Agent

| | | |
|---|---|---|
| Thioglycolic acid (80% aqueous solution) | 9.5 | wt. % |
| Ammonia (25% aqueous solution) | 1.6 | wt. % |
| Ammonium carbonate | 4.5 | wt. % |
| Natural zein | 2.0 | wt. % |
| Perfume oil | 0.2 | wt. % |
| Water | to 100.0 | wt. % |

### Example 5

### Permanent Wave Fixative

| | | |
|---|---|---|
| Hydrogen peroxide | 4.6 | wt. % |
| Citric acid | 0.2 | wt. % |
| Natural Zein | 2.0 | wt. % |
| Perfume oil | 0.1 | wt. % |
| Water | to 100.0 | wt. % |

1. Use of natural zein for improving the condition of hair.

2. Use according to claim 1, for hardening, strengthening, restructuring, repairing or stabilizing hair or for increasing the luster, volume or combability of hair.

3. Use according to claim 1 or 2 on weakened and/or damaged hair.

4. Use according to one of claims 1 to 3, characterized in that the agent is brought in contact with the hair during or after exposure of the hair to chemical and/or physical noxae.

5. Use according to claim 4 in a pretreatment agent before a chemical and/or physical treatment of hair.

6. Use according to one of claim 1 to 5 to protect from damage, or reduce damage to, the internal structure of hair or to repair (restructure) hair.

7. Use according to claim 5, characterized in that the chemical treatment comprises oxidative dyeing, bleaching or permanent deformation.

8. Use according to one of the preceding claims for improving the hair condition of sensitive, brittle and/or fine hair.

9. Use according to one of the preceding claims to increase the volume or the fullness of hair.

10. Use according to on of claims 1 to 9, characterized in that the agent contains natural zein in an amount from 0.001 to 20.0 weight percent, based on the total amount.

11. Use according to one of claims 1 to 10, characterized in that the agent contains natural zein in an amount from 0.05 to 3.0weight percent based on the total amount.

12. Use according to one of claims 1 to 11, characterized in that the cosmetic agent is a solution, emulsion, foam, cream or gel.

13. Use according to one of claim 1 to 12, characterized in that the agent is brought in contact with the hair for a duration of 1 to 60 minutes at a temperature between 20°C. and 60° C.

14. Permanent wave agent containing
(A) natural zein and
(B) at least one hair keratin-reducing substance.

15. Agent for oxidative treatment of hair containing
(A) natural zein and
(B) at least one oxidant.

16. Agent according to claim 15, characterized in that the oxidant is selected from among potassium bromate, sodium bromate, sodium perborate, dehydroascorbic acid, hydrogen peroxide and urea peroxide.

17. Hair colorant containing
(A) natural zein and
(B) one or more oxidation dye precursors.
